(19) United States
(12) Patent Application Publication
(10) Pub. No.: US 2013/0267905 A1
(43) Pub. Date: Oct. 10, 2013

(54) NON-DETACHABLE RESERVOIR HOLDER FOR A DRUG DELIVERY DEVICE

(71) Applicants: Axel Teucher, Frankfurt am Main (DE); Michael Jugl, Frankfurt am Main (DE); Thomas Frederick Osman, Warwickshire (GB); David Sanders, Warwickshire (GB); Richard James Vincent Avery, Gloucestershire (GB); Joseph Butler, Warwickshire (GB)

(72) Inventors: Axel Teucher, Frankfurt am Main (DE); Michael Jugl, Frankfurt am Main (DE); Thomas Frederick Osman, Warwickshire (GB); David Sanders, Warwickshire (GB); Richard James Vincent Avery, Gloucestershire (GB); Joseph Butler, Warwickshire (GB)

(21) Appl. No.: 13/825,142

(22) PCT Filed: Sep. 28, 2011
(86) PCT No.: PCT/EP11/66930

§ 371 Date: Jun. 17, 2013

(30) Foreign Application Priority Data
Dec. 13, 2010 (EP) 10194724.0

(51) Int. Cl. A61M 5/315

(52) U.S. Cl. CPC A61M 5/31535
USPC 604/189; 604/207

(57) ABSTRACT

Disclosed herein are various examples of a drug delivery system having a non-detachable reservoir holder. The drug delivery system includes a dose setting mechanism and a reservoir holder attached to the dose setting mechanism, wherein the reservoir holder is configured to partially detach from the dose setting mechanism to allow for (i) insertion of a reservoir into the reservoir holder without being fully detached from the dose setting mechanism and (ii) removal of the reservoir from the reservoir holder without being fully detached from the dose setting mechanism.

### CROSS REFERENCE TO RELATED APPLICATIONS

The present application is a U.S. National Phase Application pursuant to 35 U.S.C. §371 of International Application No. PCT/EP2011/066930 filed Sep. 28, 2011, which claims priority to U.S. Provisional Patent Application No. 61/388,042 filed Sep. 30, 2010 and European Patent Application No. 10194724.0 filed Dec. 13, 2010. The entire disclosure contents of these applications are herewith incorporated by reference into the present application.

### FIELD OF DISCLOSURE

The present disclosure is generally directed to reservoirs, particularly reservoirs containing a medicament. More particularly, it is generally directed to a drug delivery device that includes a non-detachable reservoir holder. As just one example, such medicament reservoirs may comprise an ampoule, a cartridge, a vial, or a pouch, and may be used with a drug delivery device. Exemplary drug delivery devices include, but are not limited to syringes, pen type injection syringes, pumps, inhalers, or other similar injection or infusing devices that require at least one reservoir containing at least one medicament.

### BACKGROUND

Medicament reservoirs such as ampoules, cartridges, or vials are generally known. Such reservoirs are especially used for medicaments that may be self administered by a patient. For example, with respect to insulin, a patient suffering from diabetes may require a certain amount of insulin to either be injected via a pen type injection syringe or infused via a pump. With respect to certain known reusable pen type drug delivery devices, a patient loads a cartridge containing the insulin into a proximal end of a cartridge holder. After the cartridge has been correctly loaded, the user may then be called upon to select a dose of medicament. Multiple doses may be dosed from the cartridge. Where the drug delivery device comprises a reusable device, once the cartridge is empty, the cartridge holder is disconnected from the drug delivery device and the empty cartridge is removed and replaced with a new cartridge. Most suppliers of such cartridges recommend that the user dispose of the empty cartridges properly. Where the drug delivery device comprises a disposable device, once the cartridge is empty, the user is recommended to dispose of the entire device.

Such known self administration systems requiring the removal and reloading of empty cartridges have certain limitations. For example, in certain generally known systems, a user simply loads a new cartridge into the delivery system without the drug delivery device or without the cartridge having any mechanism of preventing cross use of an incorrect cartridge. That is, the drug delivery device does not have a mechanism for determining if the medicament contained in the cartridge is indeed the correct type of medicament to be administered by the patient. Alternatively, certain known drug delivery devices do not present a mechanism for determining if the correct type of medicament within the cartridge should be used with that particular drug delivery system. This potential problem could be exacerbated given that certain elderly patients, such as those suffering from diabetes, may have limited manual dexterity. Identifying an incorrect medicament is quite important, since the administration of a potentially incorrect dose of a medicament such as a short acting insulin in lieu of a long insulin could result in injury or even death.

Some drug delivery devices or systems may use a color coding scheme to assist a user or care giver in selecting the correct cartridge to be used with a drug delivery device. However, such color coding schemes pose challenges to certain users, especially those users suffering from poor eyesight or color blindness: a situation that can be quite prevalent in patients suffering from diabetes.

Another concern that may arise with such disposable cartridges is that these cartridges are manufactured in essentially standard sizes and manufactured to comply with certain recognized local and international standards. Consequently, such cartridges are typically supplied in standard sized cartridges (e.g., 3 ml cartridges). Therefore, there may be a variety of cartridges supplied by a number of different suppliers and containing a different medicament but they may fit a single drug delivery device. As just one example, a first cartridge containing a first medicament from a first supplier may fit a drug delivery device provided by a second supplier. As such, a user might be able to load an incorrect medicament (such as a rapid or basal type of insulin) into a drug delivery device and then dispense the medicament without being aware that the drug delivery device was perhaps neither designed nor intended to be used with such a cartridge.

As such, there is a growing desire from users, health care providers, care givers, regulatory entities, and medical device suppliers to reduce the potential risk of a user loading an incorrect drug type into a drug delivery device. There is also, therefore, a desire to reduce the risk of dispensing an incorrect medicament (or the wrong concentration of the medicament) from such a drug delivery device.

There is, therefore, a general need to physically dedicate or mechanically code a cartridge to its drug type and design an injection device that only accepts or works with the dedication or coded features provided on or with the cartridge so as to prevent unwanted cartridge cross use. Similarly, there is also a general need for a dedicated cartridge that allows the drug delivery device to be used with only an authorized cartridge containing a specific medicament while also preventing undesired cartridge cross use.

Further, there is a general need to ensure that only given cartridge holders intended for given cartridges be used with given drug delivery devices.

There is also a general need to provide a dedicated cartridge that is difficult to tamper with so that the cartridge may not be compromised in that the cartridge can be used with an unauthorized drug or drug delivery device. Because such cartridges may be difficult to tamper with, they may also reduce the risk of counterfeiting by making it more difficult for counterfeiters to provide unregulated counterfeit medicament carrying products.

It is an aim to provide a drug delivery system which comprises an improved holder for the cartridge.

### SUMMARY

This aim can be achieved by a drug delivery system according to claim 1. According to an exemplary arrangement, a drug delivery system includes a non-detachable reservoir holder. In particular, the drug delivery system includes a dose setting mechanism and a reservoir holder attached to the dose setting mechanism, wherein the reservoir holder is configured to partially detach from the dose setting mechanism, preferably in an axial direction, to allow for (i) insertion of a reservoir into the holder without being fully detached from the dose setting mechanism and (ii) removal of the reservoir from the holder without being fully detached from the dose setting mechanism. “Partially detaching” means that the reservoir holder is configured to move away from the dose setting mechanism or is moved in the distal direction with respect to the dose setting mechanism wherein the reservoir holder is not fully detached. In an example, the reservoir holder may include a coding feature that prevents incorrect reservoirs from being fully inserted in the reservoir holder.

The reservoir holder may comprise an opening at a proximal end for insertion of the reservoir and removal of the reservoir. Alternatively, the reservoir holder comprises an opening in a sidewall of the reservoir holder for insertion of the reservoir and removal of the reservoir, wherein an inner surface of the sidewall may comprise a coding feature. In one embodiment an inner surface of the reservoir holder comprises a coding feature.

In one embodiment the dose setting mechanism comprises a detachable snap connection feature for detachably fixing the reservoir holder to the dose setting mechanism. The reservoir holder may comprise an elongated hole connection feature configured to detachably connect to the detachable snap connection feature. Alternatively, the reservoir holder may comprise a detachable snap connection feature and the dose setting mechanism may comprise an elongated hole connection feature. When a user partially detaches the reservoir holder from the dose setting mechanism, the detachable snap connection feature moves distally within the elongated hole connection thus freeing space for a user to remove or insert the reservoir. Preferably, the drug delivery system has a reservoir holder which comprises an opening in a sidewall of the reservoir holder for insertion of the reservoir and removal of the reservoir.

In one embodiment the reservoir holder comprises a proximal end having a diameter greater than a distal end of the dose setting mechanism.

One embodiment of the drug delivery system comprises a transverse hinge feature, wherein the transverse hinge feature connects the drug reservoir holder and the dose setting mechanism. In one embodiment the transverse hinge feature comprises a sliding joint. The reservoir holder may be configured to partially detach from the dose setting mechanism in an axial direction, and is further configured to rotate relative to the dose setting mechanism after being partially detached. The reservoir holder may comprise an opening at a proximal end and wherein the reservoir is insertable in the proximal end.

In one embodiment the drug delivery system further comprises a flexible connector feature. The flexible connector feature may comprise a strap holding a portion of the flexible connector feature to a distal end of the dose setting mechanism. In one embodiment the flexible connector is composed of an injection-molded polymer such as PP, HDPE, or PA. The flexible connector feature comprises a wire connecting the reservoir holder to the dose setting mechanism.

One embodiment of the drug delivery system further comprises a collar which incorporates a bayonet and which is attached to the distal end of the dose setting mechanism, the reservoir holder incorporating a locking pin near its proximal end.

These as well as other advantages of various aspects of the present invention will become apparent to those of ordinary skill in the art by reading the following detailed description, with appropriate reference to the accompanying drawings.

### BRIEF DESCRIPTION OF THE DRAWINGS

Exemplary embodiments are described herein with reference to the drawings, in which:

FIG. 1 illustrates an exemplary embodiment of a pen type drug delivery device;

FIG. 2 illustrates an exemplary embodiment of a drug cartridge;

FIG. 2a illustrates an exemplary embodiment of a drug delivery device having a non-detachable reservoir holder;

FIG. 2b illustrates the example drug delivery device of FIG. 2a during insertion/removal of a reservoir;

FIG. 2c is a cross-sectional view of FIG. 2b;

FIG. 2d illustrates the example drug delivery device of FIGS. 2a-c in a ready-to-use configuration;

FIG. 3 illustrates another exemplary embodiment of a drug delivery device having a non-detachable reservoir holder;

FIG. 4 illustrates another exemplary embodiment of a drug delivery device having a non-detachable reservoir holder;

FIG. 5 illustrates another exemplary embodiment of a drug delivery device having a non-detachable reservoir holder;

FIG. 6 illustrates another exemplary embodiment of a drug delivery device having a non-detachable reservoir holder; and

FIG. 7 illustrates an embodiment of a dedicated cartridge holder and corresponding cartridge.

### DETAILED DESCRIPTION

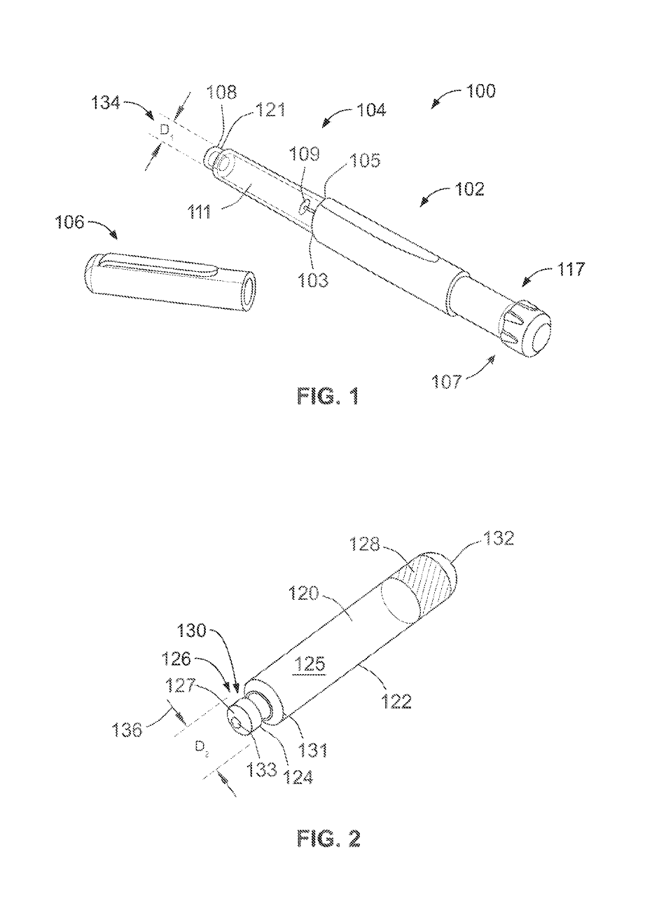

Disclosed herein are various exemplary drug delivery devices or drug delivery systems that include various embodiments of non-detachable reservoir holders. Herein, reservoirs and reservoir holders are sometimes referred to as “cartridges” and “cartridge holders” respectively. FIG. 1 shows an exemplary drug delivery device 100 in the form of a pen type syringe that may include a non-detachable reservoir holder or cartridge holder 104 in accordance with the proposed concept. Drug delivery device 100 comprises a dose setting mechanism 102, a cartridge holder 104, and a removable cap 106. A proximal end 105 of the cartridge holder 104 and a distal end 103 of the dose setting mechanism 102 may be removably secured together, perhaps with threads (or other suitable connecting mechanism such as a snap lock, press fit, or bayonet lock mechanism), or may be permanently secured together, perhaps with adhesive.

The drug delivery device 100 may comprise a re-usable or a disposable pen type syringe. As shown in FIG. 1, the dose setting mechanism 102 comprises a spindle (or piston rod) 109, such as a threaded spindle 109 that rotates when a dose is injected. In other embodiments, the spindle 109 may not rotate upon dose injection.

To inject a previously set dose, a double ended needle assembly (not shown) is attached to a distal end 108 of the cartridge holder 104. As shown, the distal end 108 of the cartridge holder 104 comprises a thread 121 (or other suitable connecting mechanism such as a snap lock, press fit or bayonet lock mechanism) so that the needle assembly may be removably attached to the distal end 108 of the cartridge holder 104. When the drug delivery device 100 is not in use, the removable cap 106 can be releasably retained over the cartridge holder 104.

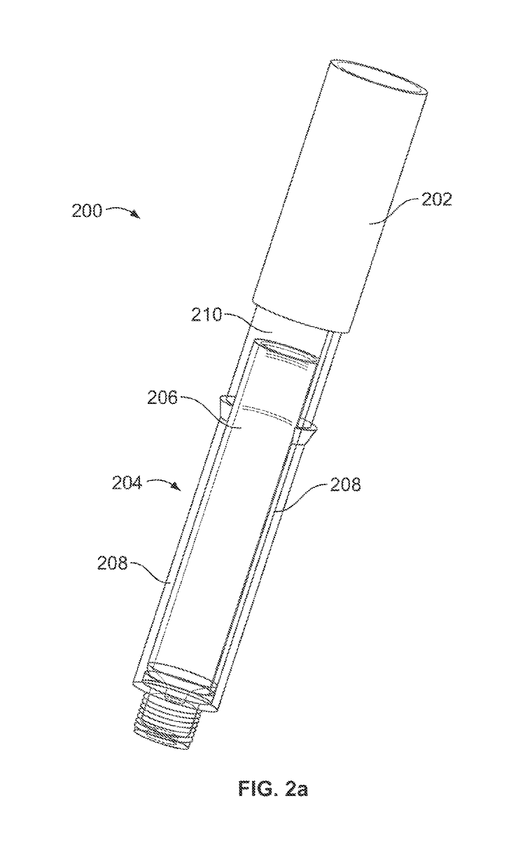

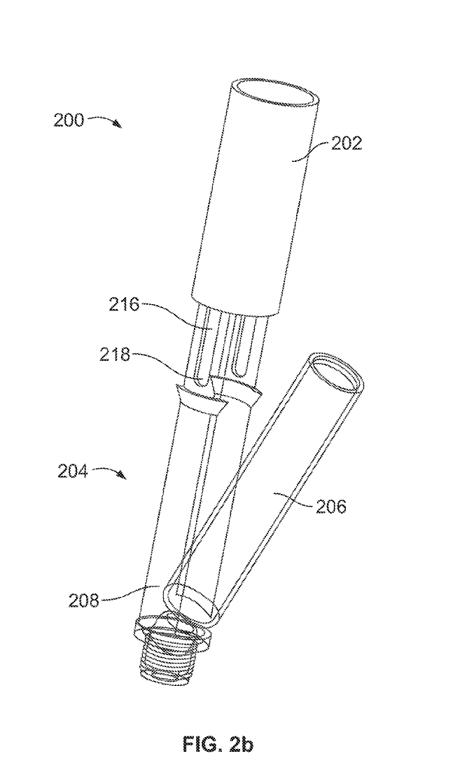

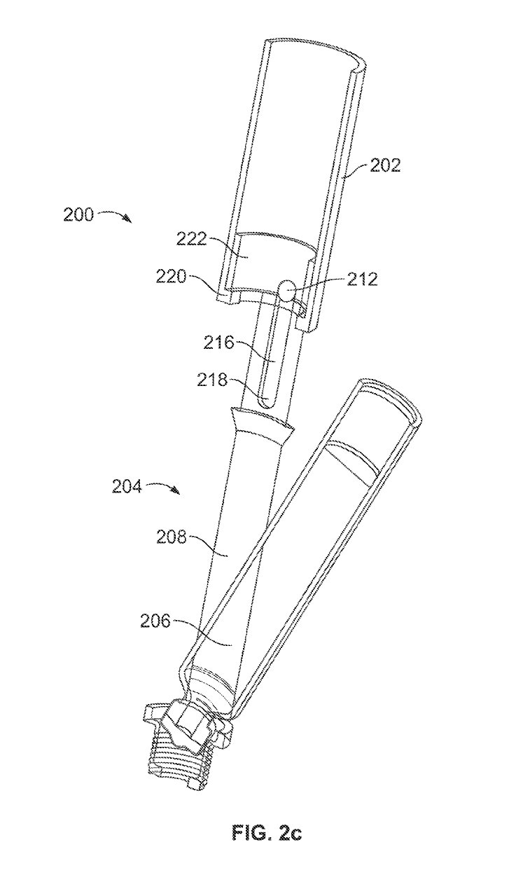

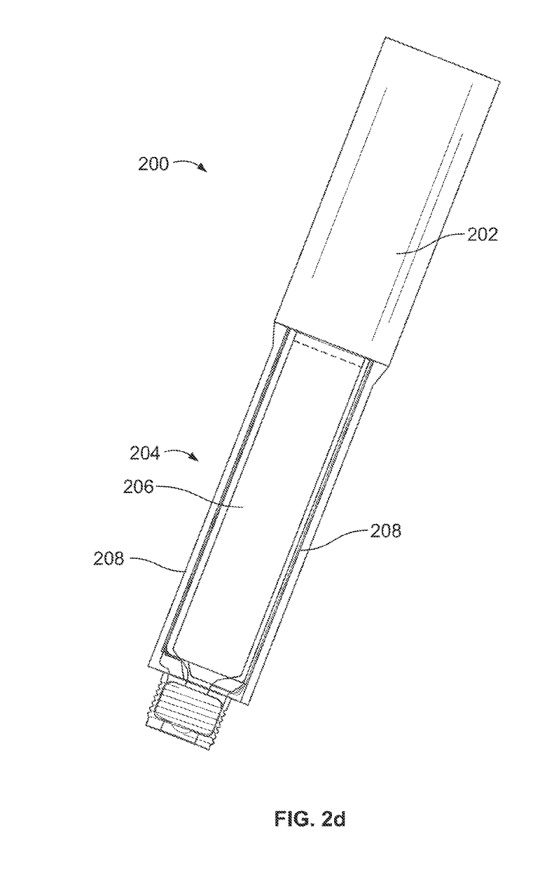

An inner cartridge cavity 111 defined by the cartridge holder 104 is dimensioned and configured to securely receive and retain a cartridge, such as cartridge 120 illustrated in FIG. 2. Cartridge 120 may be composed of various materials including glass. As shown, cartridge 120 has a generally tubular barrel 122 extending from a distal end 130 to a proximal end 132.

At the distal end 130, cartridge 120 includes a smaller diameter neck 126 projecting distally from the shoulder 131 of the barrel 122. The smaller diameter neck 126 is provided with a large diameter annular bead 124 that extends circumferentially thereabout at the distal end of the neck 126 and defines an opening 127. A pierceable seal or septum 133 is securely held across the opening 127 by a metallic sleeve or a ferrule.

The medicament 125 is pre-filled into the cartridge 120 and is retained within the cartridge 120, in part, by the pierceable seal or septum 133, a ferrule, and a stopper (also commonly referred to as a “piston” or a “bung”) 128. The stopper 128 is arranged at the proximal end 132 of the cartridge 120 and is in sliding fluid-tight engagement with the inner tubular wall of the barrel 122. Axially directed forces created by the spindle 109 of the dose setting mechanism 102 act upon the stopper 128 during dose injection or dose administration and urge the medicament 125 from the cartridge 120 through a double ended needle mounted onto the distal end 108 of the cartridge holder 104, and into the injection site.

A portion of the cartridge holder 104 defining the cartridge holder cavity 111 is of substantially uniform diameter represented in FIG. 1 by D1 134. Diameter D1 134 is preferably slightly greater than the diameter D2 136 of the cartridge 120. The interior of the cartridge holder 104 includes an inwardly-extending annular portion or stop that is dimensioned to prevent the cartridge 120 from moving within the cartridge holder 104. In this manner, when the cartridge 120 is loaded into the cavity 111 of the cartridge holder 104 and the cartridge holder 104 is then connected to the dose setting mechanism 102, the cartridge assembly 120 will be securely held within the cartridge cavity 111.

A number of doses of medicament 125 may be dispensed from the cartridge 120. It will be understood that the cartridge 120 may contain a type of medicament 125 that must be administered often, such as one or more times a day. One such medicament 125 is insulin.

The dose setting mechanism 102 comprises a dose setter 117 at its proximal end 107. In one preferred arrangement, the dose setter 117 may extend along the entire length of the dose setting mechanism 102. The dose setter 117 may be rotated by a user so as to set a dose.

To administer a dose, the user attaches a needle assembly comprising a double ended needle on the distal end 108 of the cartridge holder 104. In this manner, the needle assembly pierces the seal 133 of the cartridge 120 and is therefore in liquid communication with the medicament 125. The user pushes on the dose setter 117 to inject the set dose. The same dose setting and dose administration procedure is followed until the medicament 125 in the cartridge 120 is expended and then a new cartridge 120 must be loaded in the drug delivery device 100. To exchange an empty cartridge 120, the user must gain access to the empty cartridge 120 by manipulating the cartridge holder 104 and/or dose setting mechanism 102.

In accordance with the disclosed concept, a reservoir holder, such as cartridge holder 104, may be non-detachable from a dose setting mechanism, such as dose setting mechanism 102. In general, a drug delivery system 100 in accordance with the proposed concept includes a dose setting mechanism 102 and a reservoir holder, such as cartridge holder 104, attached to the dose setting mechanism 102, where the reservoir holder 104 is configured to partially detach from the dose setting mechanism 102 to allow for (i) insertion of a reservoir, such as cartridge 120, into the reservoir holder 104 without being fully detached from the dose setting mechanism 102 and (ii) removal of the reservoir from the reservoir holder without being fully detached from the dose setting mechanism 102. Further, the reservoir holder 104 may comprise a coding feature that prevents incorrect reservoirs from being inserted in the reservoir holder 104.

In such a drug delivery system, the reservoir holder or cartridge holder 104 can be withdrawn from the drug delivery device 100 sufficiently to change the reservoir 120, but the reservoir holder 104 remains attached to the drug delivery device 100 (i.e., the reservoir 120 is not fully detachable). As mentioned above, a reservoir or cartridge 120 may be coded to the reservoir holder 104; however, since the reservoir holder 104 is not fully detachable from the drug delivery device 100, the reservoir holder 104 does not need to be separately coded to the device 100. This may minimize reservoir holder mix-ups, as a user will be forced to use the proper holder 104 with the drug delivery device 100. Further, this system may prevent the reservoir holder 104 from being lost or misplaced. Still further, since the holder 104 may be coded to the reservoir 120, only given reservoirs 120 may be used with the drug delivery device 100, which may help to prevent unwanted reservoir cross use. In an example, it may not be possible to fully insert an incorrect reservoir 120 into the non-detachable holder 104. Example non-detachable reservoir holders 104 that may be coded to respective reservoirs 120 are discussed below with reference to FIGS. 2a-6.

FIGS. 2a-d depict a first example drug delivery device 200 comprising a non-detachable reservoir holder 204 and a dose setting mechanism 202. It should be understood that “non-detachable” is intended to indicate that the reservoir holder 204 may not be fully detached from the device 200, although it may be partially detached in order to allow for insertion and removal of a reservoir 206.

FIG. 2a shows drug delivery device 200 wherein non-detachable reservoir holder 204 and dose setting mechanism 202 are partially detached and where a reservoir 206 is inserted in reservoir holder 204. As shown, reservoir holder 204 comprises a main body having sidewall 208 and an opening 210 through the sidewall 208 that acts as an access point that allows reservoir 206 to be inserted and removed from reservoir holder 204 when reservoir holder 204 is partially detached from dose setting mechanism 202.

Turning to FIGS. 2b and 2c, to facilitate (i) the insertion and removal of reservoir 206 from reservoir holder 204 and (ii) the fixation of the reservoir holder 204 to the dose setting mechanism 202, drug delivery device 200 includes a detachable snap connection feature 212 near the distal end of the dose setting mechanism 202 and an elongated hole connection feature 216 near the proximal end of the reservoir holder 204. However, other detachable connection features are possible as well, such as a bayonet or screwthread. Moreover, it should be understood that in other embodiments the snap connection feature 212 may be part of the reservoir holder 204 and the elongated hole feature 216 may be part of the dose setting mechanism 202.

After reservoir 206 is inserted into reservoir holder 204, holder 204 is fixed to dose setting mechanism 202 by using snap connection feature 212. This is accomplished by bringing the reservoir holder 204 and the dose setting mechanism 202 together until the snap connection feature 212 snaps into the distal end 218 of the elongated hole connection feature 216. The drug delivery device 200 is now in its ready-for-use configuration (i.e., dose administration configuration). To accomplish the snap connection, the width of the elongated hole connection feature 216 near its distal end 218 may by less than the width (or diameter) of the snap connection feature 212 (e.g., there may be inwardly projecting features on either side, or both sides, of the elongated hole connection feature 216).

FIG. 2d illustrates drug delivery device 200 in its ready-for-use configuration. Accordingly, reservoir holder 204 is firmly fixed, via detachable snap connection feature 212, to dose setting mechanism 202. Reservoir 206 is firmly held in the drug delivery device 200 such that it is not able to slide axially in the body of reservoir holder 204. However, there may be some axial play. Such axial play may be the result of geometric tolerances.

Turning back to FIGS. 2b and 2c, after drug delivery device 200 has been used and reservoir 206 needs to be replaced, a user can remove reservoir 206 by pulling the reservoir holder 204 in the distal direction and/or by pulling the dose setting mechanism 202 in the proximal direction, and, under a given force, the snap connection feature 212 will release from the elongated hole connection feature 216. This release allows the reservoir holder 204 to move in the distal direction thus partially detaching the holder 204 from the dose setting mechanism 202.

When a user partially detaches the reservoir holder 204 from the dose setting mechanism 202, a user may remove the reservoir 206. In addition to the snap feature 212 preventing full detachment of the dose setting mechanism 202 from the reservoir holder 204, the proximal end (or plug) 222 of the holder 204 has a diameter greater than the distal end 220 of the dose setting mechanism 202. Thus the plug 222 acts as a stop to prevent the reservoir holder 204 from being fully detached from the dose setting mechanism 202. That is, the plug 222 allows the partially detached reservoir holder 204 to reach a distal end position (i.e., fully extended position) as shown in FIGS. 2c and 2d. In this distal end position, reservoir 206 can be removed laterally and exchanged with a new reservoir 206. After the reservoir exchange is complete, reservoir holder 204 can be slid back proximally into the dose setting mechanism 202 until the snap connection feature 212 snaps into the distal end 218 of the elongated hole connection feature 216 and thus the new reservoir 206 is fixed in the drug delivery device 200 and the drug delivery device 200 is ready for use.

In addition to the above-mentioned features, the reservoir holder 204 may include a coding feature that serves to only allow given types of reservoirs 206 to be inserted in reservoir holder 204 and thus be used with drug delivery device 200.

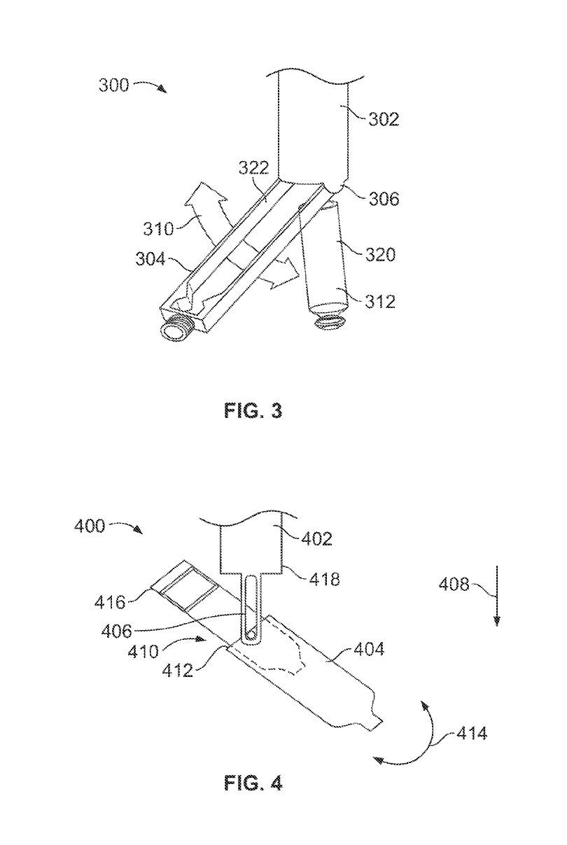

Other types of non-detachable reservoir holders that can be partially detached but not fully detached from a dose setting mechanism are possible as well. FIG. 3 depicts a second example drug delivery device 300 comprising a dose setting mechanism 302 and a non-detachable cartridge holder 304 attached to one another with a transverse hinge 306. The transverse hinge 306 allows a user to rotate the holder 304 relative to the body of the dose setting mechanism 302. In an example, a user may be able to rotate the holder 304 in a direction depicted by arrow 310. When the cartridge 312 is rotated from the axially aligned position, the user may be able to insert or remove a cartridge, such as cartridge 312. In this example and similar to the example depicted in FIG. 2, the user may insert the cartridge 312 into the holder 304 via a side hole. During use, the cartridge holder 304 is aligned axially with the dose setting mechanism 302 and securely holds the cartridge 312 in the drug delivery device 300.

In an embodiment, the transverse hinge may include a sliding joint. A sliding joint may allow a cartridge to be fitted axially in the holder, rather than laterally. An example transverse hinge with a sliding joint is shown in FIG. 4. Drug delivery device 400 includes a dose setting mechanism 402 and a non-detachable cartridge holder 404 attached to one another via a transverse hinge that has a sliding joint 406. The cartridge holder 404 is configured to partially detach from the dose setting mechanism 402 in an axial direction 408 and is further configured to rotate relative to the dose setting mechanism 402 after being partially detached. For example, the cartridge holder 404 may rotate in the direction indicated by arrow 414. Since the proximal end 412 of the holder 404 can be removed from distal end 418 of the dose setting mechanism 402 and rotated, a cartridge 416 may be inserted in an opening 410 at proximal end 412.

After a user inserts a cartridge 416 in the cartridge holder 404, the user may rotate the cartridge holder 404 to align with the dose setting mechanism 402 and may reconnect the holder 404 to the dose setting mechanism 402. Similar to the example of FIGS. 2a-2d, the drug delivery device 400 may include a detachable connection feature (e.g., snap connection feature, screwthread or bayonet).

In another example, a drug delivery device 400 may include a flexible connection feature that connects the dose setting mechanism 402 and the reservoir holder 404. A flexible connection feature may allow a user to rotate a reservoir holder 404 in the X-axis, Y-axis, and Z-axis, thus giving the user freedom to move the holder into any position the user would like in order to insert or remove a cartridge 416.

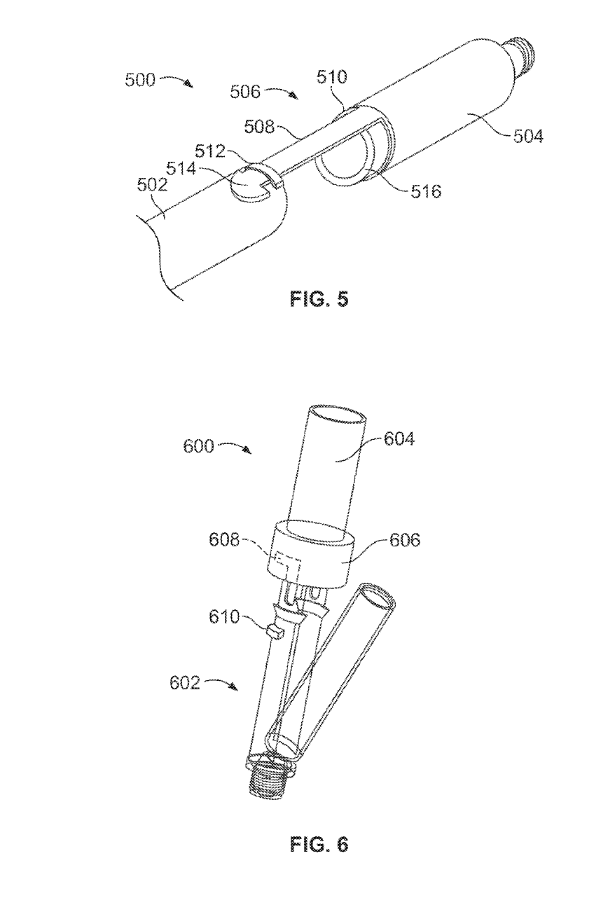

FIG. 5 depicts an example drug delivery device 500 having a flexible connection feature 506. In particular, drug delivery device 500 includes a dose setting mechanism 502 and a non-detachable cartridge holder 504 attached to one another with a flexible connector feature 506. In this example, the flexible connector feature 506 is securely fastened to both the cartridge holder 504 and dose setting mechanism 502. The flexible connector feature 506 includes a strip 508 of flexible material that is attached to both the cartridge holder 504 and dose setting mechanism 502. The strip 508 may be coupled to the cartridge holder 504 and dose setting mechanism 502 in any desired way. In the example of FIG. 5, the cartridge holder 504 can rotate relative to the strip 508, allowing the cartridge holder 504 to be securely fixed to the dose setting mechanism 502 using a connection feature such as a bayonet, or screwthread. Additionally, in the example of FIG. 5, a strap 512 secures the strip 508 to the dose setting mechanism 502. The strap 512 allows the strip 508 to slide axially. FIG. 5 depicts the strip in a distal end position, where a hook 514 of the connector feature 506 hooks onto the strap 512, thereby prevent further distal movement. However, a user may rotate the holder 504 in the X-axis, Y-axis, and Z-axis to insert or remove a cartridge, such as cartridge 516.

The flexible connector feature 506 may be composed of various materials. Example materials include but are not limited to injection-molded polymers such as PP, HDPE, PA. In another example, the flexible connector feature 506 may be a wire that holds the dose setting mechanism 502 to the cartridge holder 504. Further, the flexible connector feature 506 may comprise a spring.

In another example drug delivery device 600 shown in FIG. 6, to lock a reservoir holder 602 to a dose setting mechanism 604, a collar 606 may slide or rotate relative to the drug delivery device 600 and reservoir holder 602. As shown, collar 606 incorporates a bayonet 608 and is attached to the distal end of the dose setting mechanism 604. The reservoir holder 602 incorporates a locking pin 610 near its proximal end. To lock the reservoir holder 602 in place, the user first pushes the reservoir holder 602 in the proximal direction, then rotates the collar 606 to lock the pin 610 within the bayonet 608.

The non-detachable holder 104, 204, 304 404, 504, 602 of any of the above-mentioned exemplary drug delivery devices 100, 200, 300, 400, 500, 600 may be coded to a given type of drug reservoir or reservoirs 120, 206, 312, 416, 516 in order to prevent unwanted reservoir cross use. In addition, the reservoir may 120, 206, 312, 416, 516 be coded to the dose setting mechanism 102, 202, 302, 402, 502, 604. In general, any method to dedicate given drug reservoirs 120, 206, 312, 416, 516 to given reservoir holders 104, 204, 304 404, 504 602 or given dose setting mechanism 102, 202, 302, 402, 502, 604 may be used. For example, a system of a coding feature on a drug cartridge 120, 206, 312, 416, 516 and a corresponding coding feature on a drug cartridge holder 104, 204, 304 404, 504, 602 may be used. That is, a drug cartridge 120, 206, 312, 416, 516 may have a coding feature disposed at a given point on the cartridge 120, 206, 312, 416, 516, such as near the proximal end 132 of the cartridge 120, 206, 312, 416, 516 or near the distal end 130. In addition, the cartridge holder 104, 204, 304 404, 504, 602 may have a corresponding coding feature, configured for accepting the cartridge coding.

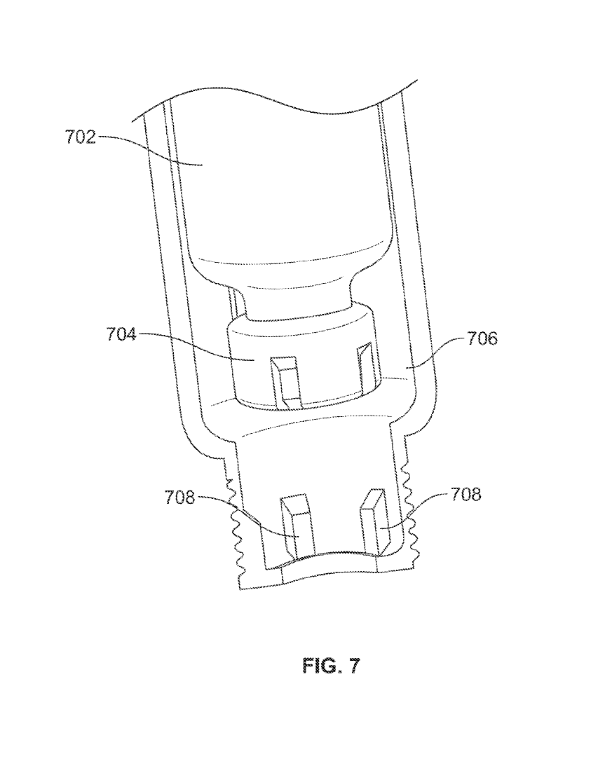

As shown in FIG. 7, in an example, a cartridge 702 includes a coded ferrule 704. Similarly, the cartridge holder 706 has a corresponding coding feature 708 disposed on the inner surface of its distal end. However, it should be understood that any type of corresponding coding features may be used. If a cartridge 702 that is not intended for the holder 706 is inserted into the holder 706, the incorrect cartridge 702 may not be able to fully inserted because the ferrule 704 or distal end of the incorrect cartridge 702 does not match the coding feature 708 of the holder 706.

In an example, with reference to FIG. 3, a coding feature (e.g., a protrusion) may be disposed on a sidewall 320 of the cartridge 312. The holder 304 may have a corresponding coding feature (e.g., an indentation) disposed on the inner sidewall 322 and configured for accepting the cartridge coding feature. In yet another example, with reference to FIG. 4 or FIG. 5, the holder 404, 504 may have a coding feature at the distal end or proximal end 510 of the holder 404, 504. Any cartridge 120, 206, 312, 416, 516, 702 inserted may need to have a corresponding coding feature 704 to be inserted into the holder 104, 204, 304 404, 504, 602, 706 and used with the drug delivery device 100, 200, 300, 400, 500, 600.

Although aimed primarily at the insulin market, the proposed system may apply to other drugs. The proposed drug delivery system results in a number of advantages. For example, since the holder 104, 204, 304 404, 504, 602, 706 is not fully detachable from the drug delivery device 100, 200, 300, 400, 500, 600, the holder 104, 204, 304 404, 504, 602, 706 does not need to be separately coded to the device 100, 200, 300, 400, 500, 600. Further, this system may prevent the holder 104, 204, 304 404, 504, 602, 706 from being lost or misplaced. Still further, since the holder 104, 204, 304 404, 504, 602, 706 may be coded to the reservoir 120, 206, 312, 416, 516, 702, only given reservoirs 120, 206, 312, 416, 516, 702 may be used with the drug delivery device 100, 200, 300, 400, 500, 600, which may help to prevent unwanted reservoir cross use.

The term “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyhepta [symbol] decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative; or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5-des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

Exemplary embodiments of the present invention have been described. The scope of protection of the invention is not limited to the examples given hereinabove. The invention is embodied in each novel characteristic and each combination of characteristics, which particularly includes every combination of any features which are stated in the claims, even if this feature or this combination of features is not explicitly stated in the claims or in the examples. However, as those of skill in the art will recognize certain changes or modifications to such arrangements may be made. Those skilled in the art will understand, however, that further changes, modifications, revisions and/or additions may be made to the presently disclosed arrangements without departing from the true scope and spirit of the present invention, which is defined by the claims.

1-17. (canceled)

18. A drug delivery system comprising:
a dose setting mechanism;
a reservoir holder attached to the dose setting mechanism, wherein the reservoir holder is configured to move away from the dose setting mechanism in an axial direction to allow for (i) insertion of a reservoir into the reservoir holder without being fully detached from the dose setting mechanism and (ii) removal of the reservoir from the reservoir holder without being fully detached from the dose setting mechanism.

19. The drug delivery system of claim 18, wherein the reservoir holder comprises a coding feature that prevents incorrect reservoirs from being fully inserted in the reservoir holder.

20. The drug delivery system of claim 18, wherein the reservoir holder comprises an opening at a proximal end for insertion of the reservoir and removal of the reservoir.

21. The drug delivery system of claim 18, wherein the reservoir holder comprises an opening in a sidewall of the reservoir holder for insertion of the reservoir and removal of the reservoir.

22. The drug delivery system of claim 18, wherein an inner surface of the reservoir holder comprises a coding feature.

23. The drug delivery system of claim 18, wherein the dose setting mechanism comprises a detachable snap connection feature for detachably fixing the reservoir holder to the dose setting mechanism or wherein the reservoir holder comprises a detachable snap connection feature.

24. The drug delivery system of claim 23, wherein the reservoir holder the dose setting mechanism comprises an elongated hole connection feature configured to detachably connect to the detachable snap connection feature.

25. The drug delivery system of claim 24, wherein, when a user partially detaches the reservoir holder from the dose setting mechanism, the detachable snap connection feature moves distally within the elongated hole connection thus freeing space for a user to remove or insert the reservoir.

26. The drug delivery system of claim 18, wherein the reservoir holder comprises a proximal end having a diameter greater than a distal end of the dose setting mechanism.

27. The drug delivery system of claim 18, further comprising a transverse hinge feature, wherein the transverse hinge feature connects the reservoir holder and the dose setting mechanism.

28. The drug delivery system of claim 27, wherein the transverse hinge feature comprises a sliding joint.

29. The drug delivery system of claim 18, wherein the reservoir holder is further configured to rotate relative to the dose setting mechanism after being partially detached.

30. The drug delivery system of claim 18, further comprising a flexible connector feature.

31. The drug delivery system of claim 30, wherein the flexible connector feature comprises a strap holding a portion of the flexible connector feature to a distal end of the dose setting mechanism.

32. The drug delivery system of claim 30, wherein the flexible connector feature is composed of an injection-molded polymer such as PP, HDPE, or PA.

33. The drug delivery system of claim 30, wherein the flexible connector feature comprises a wire connecting the reservoir holder to the dose setting mechanism.

34. The drug delivery system of claim 18, further comprising a collar which incorporates a bayonet and which is attached to the distal end of the dose setting mechanism, wherein the reservoir holder incorporates a locking pin near its proximal end.
